(19) United States
(12) Patent Application Publication
(10) Pub. No.: US 2024/0415749 A1
(43) Pub. Date: Dec. 19, 2024

(54) COSMETIC SWEAT MANAGEMENT COMPOSITIONS

(71) Applicant: Conopco, Inc., d/b/a UNILEVER, Englewood Cliffs, NJ (US)

(72) Inventors: Martin Peter Cropper, Birkenhead (GB); Neil Robert Fletcher, Irby, Wirral (GB); Kristina Mervic, Nova Gorica (SI); Veronique Sandrine Munier, Liverpool (GB)

(21) Appl. No.: 18/718,976

(22) PCT Filed: Dec. 9, 2022
(86) PCT No.: PCT/EP2022/085132

§ 371 Date: Jun. 12, 2024

(30) Foreign Application Priority Data
Dec. 22, 2021 (EP) 21217116.9

(51) Int. Cl. A61K 8/34; A61K 8/04; A61K 8/31; A61K 8/37; A61Q 15/00

(52) U.S. Cl. CPC A61K 8/342; A61K 8/046; A61K 8/31; A61K 8/375; A61Q 15/00; A61K 2800/30; A61K 2800/34

(57) ABSTRACT

A cosmetic composition comprising a lipid mixture consisting of isostearyl alcohol and glycerol monolaurate at a weight ratio of from 25:75 to 40:60 and a fatty material selected from C10-C18 linear alcohols and C8 to C16 alkanes, and the use of such compositions in sweat management.

### FIELD OF INVENTION

The present invention is in the field of cosmetic compositions and their use as sweat management compositions, in particular, non-aluminium sweat management compositions.

### BACKGROUND

EP 550,960 A1 (Unilever, 1992) discloses the use as an antiperspirant active of a lipid mixture which forms, upon contact with perspiration, a water-insoluble liquid crystal phase of greater than one dimensional periodicity. This publication does not disclose ethanolic compositions, nor their stability issues, nor their use in aerosol compositions.

WO 94/024993 (Unilever, 1994) discloses an antiperspirant composition comprising a lipid mixture which forms, upon contact with perspiration, a water-insoluble liquid crystal phase of greater than one dimensional periodicity, in a cosmetic vehicle comprising a volatile silicone and containing less than 10% by weight of the total composition of a short chain monohydric alcohol. This publication does not disclose compositions with the specific ratios of components as defined herein and does not disclose their importance in the low temperature (0° C.) stability thereof.

WO2020/108882 A1 (Unilever, 2020) discloses antiperspirant aerosol compositions comprising ethanol, amphiphilic material similar to that disclosed in the above publications and volatile silicone, the ratios and amounts of the components being selected to provide good storage stability for the aerosols, particularly at low temperatures.

WO2020/108885 A1 (Unilever, 2020) discloses antiperspirant compositions comprising ethanol, isostearyl alcohol, glycerol monolaurate, volatile silicone and humectant, the ratios and amounts of the components being selected to provide good storage stability.

EP 3,773,433 1 (Unilever, 2021) discloses antiperspirant compositions comprising ethanol, isostearyl alcohol, glycerol monolaurate and volatile silicone, the ratios of the components being selected to provide good storage stability, particularly at low temperatures.

### SUMMARY OF INVENTION

It is an object of the present invention to provide a cosmetic aerosol composition that does not require the presence of an aluminium salt to deliver a sweat management benefit. It is a further object of the present invention to do this from a composition that has a high degree of storage stability, particularly at low temperatures.

It is an object of the present invention to provide a cosmetic aerosol composition that is free from silicone oil and which has good stability, particularly at low temperature. It is a further object of the present invention to provide such a cosmetic aerosol composition that does not require the presence of an aluminium salt to deliver a sweat management benefit.

In a first aspect of the invention, there is provided a cosmetic composition comprising:
(a) a lipid mixture consisting of isostearyl alcohol and glycerol monolaurate at a weight ratio of from 25:75 to 40:60 at a level of from 2.5% to 25% by weight; and
(b) a fatty material selected from C10-C18 linear alcohols and C8 to C16 alkanes; wherein the ratio of component (a) to total components (b) is from 73:27 to 88:12 by weight; with the additional requirements that:
(i) when 1-decanol and/or 1-dodecanol is the major component (b) present, the ratio of component (a) to component (b) is from 81:19 to 88:12;
(ii) when stearyl alcohol and/or a C8 to C16 alkane is the major component (b) present, the ratio of component (a) to component (b) is from 78:22 to 88:12;
   wherein the amounts indicated exclude any volatile propellant present in the composition.

In a second aspect of the invention, there is provided a method of manufacture of a cosmetic aerosol composition according to the first aspect of the invention.

In a third aspect of the invention, there is provided a cosmetic method of reducing wetness perception comprising the topical application of a composition according to the first aspect of the invention.

### DETAILED DESCRIPTION

Herein, features expressed as “preferred” with regard to a particular aspect of the invention should be understood to be preferred with regard to each aspect of the invention (likewise, features expressed as “more preferred” or “most preferred”).

Herein, preferred features of the invention are particularly preferred when used in combination with other preferred features.

Herein, “ambient conditions” refers to 25° C. and 1 atmosphere pressure, unless otherwise indicated.

Herein, the term “volatile” refers to a material having a boiling point of less 10° C.

Herein, all percentages, ratios and amounts are by weight, unless otherwise indicated.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Herein, “water-insoluble” means having a solubility in water of less than 0.1% by weight (at 37° C.).

Herein, a “major component” is one that comprises greater than 50% by weight of the components indicated and preferably from 50.1 to 100% of such components.

The compositions of the invention are particularly effectively when applied to the underarm regions of the human body and/or the feet. The compositions are especially effectively when applied to the underarm regions of the human body.

Cosmetic aerosol compositions consist of a propellant and a base. The components of the base are typically mixed together first and the propellant is added last in a process sometimes called “gassing”.

Herein, the “base” of a cosmetic aerosol composition is all the components of the total composition other than the propellant.

It is important that the fully formulated cosmetic aerosol composition has good storage stability, so that it can survive prolonged transit to stores and extended periods on shelf prior to purchase and use.

The present invention involves compositions having superior storage stability, particularly at low temperatures, such as 0° C. By achieving stability at low temperatures, the present invention enables effective transit and storage of the claimed compositions through cold regions of the world. In addition, it enables transit and storage at refrigerated temperatures as may be required for the preservation of heat sensitive ingredients of the composition, such as certain fragrance components.

The lipid mixture is a mixture of ISA and GML at a weight ratio of from 25:75 to 45:55. This lipid mixture is able to form an inverse hexagonal phase following contact with sweat on the surface of the human body and this leads to the sweat management benefit of the compositions of the invention (as disclosed in EP 550,960 A1 [Unilever, 1992]).

The content of lipid mixture in the composition, excluding any propellant therein, is preferably at least 4%, more preferably at least 5% and most preferably at least 10%. The content of lipid mixture, again ignoring and propellant in the composition, is preferably 30% or less and more preferably 18% or less and most preferably 15% or less, these upper amounts being combinable with each of the lower ranges indicated in this paragraph to give preferred ranges of incorporation

Most prior art compositions comprising a lipid mixture used as an antiperspirant including a volatile silicone oil as a carrier oil. The present invention negates the need for such an oil and employs a fatty material which is a linear alcohol or an alkane, both as specified, instead. Linear alcohols and alkanes as used in the present invention are believed to have sustainability advantages over the formerly employed silicone oils.

The fatty material may be a linear alcohol (C10-C18) and/or an alkane (C8-C16) with the requirements that:
(i) when component (b) is solely 1-decanol and/or 1-dodecanol, the ratio of component (a) to component (b) is from 81:19 to 88:12;
(ii) when component (b) is solely stearyl alcohol and/or a C8 to C16 alkane, the ratio of component (a) to component (b) is from 78:22 to 88:12;
   wherein the amounts indicated exclude any volatile propellant present in the composition.

The fatty material is incorporated in compositions at a preferred level of from 1 to 20%, preferably 1 to 10% and most preferably 1.5 to 5%, these levels excluding any volatile propellant that may be present in the composition.

Preferred fatty materials are liquids under ambient conditions.

Preferred linear fatty alcohols are primary fatty alcohols, often designated by the prefix “1-” in chemical nomenclature. Particularly referred linear fatty alcohols are 1-decanol, 1-dodecanol, 1-tetradecanol (myristyl alcohol), 1-hexadecanol (cetyl alcohol) and 1-octadecanol (stearyl alcohol), and mixtures thereof.

The linear fatty alcohol is included in compositions of the invention such that_ratio of ISA+GML to the linear fatty alcohol is from 73:27 to 88:12; with the additional requirements that when the fatty alcohol is 1-decanol and/or 1-dodecanol, the ratio of lAS+GML to the fatty alcohol is from 81:19 to 88:12; and when the fatty alcohol is stearyl alcohol, the ratio of ISA+GML to fatty alcohol is from 78:22 to 88:12.

By having the ratio of ISA+GML to linear fatty alcohol set within the limits indicated in the above paragraph, the present inventors have found that inverse hexagonal phase may be formed at certain water levels, such as may be found on the surface of the human body as a result of sweat secretion. The production of inverse hexagonal phase can result in sweat management benefits, as described in the prior publications mentioned earlier.

The linear fatty alcohol is incorporated in compositions at a preferred level of from 1 to 20%, preferably 1 to 10% and most preferably 1.5 to 5%, these levels excluding any volatile propellant that may be present in the composition.

Preferred fatty alcohols are liquids under ambient conditions.

When alkanes are used as one of the fatty materials, they are of chain length C8 to C16, preferably C10 to C16 and more preferably C12 to C16.

Preferred alkanes for use in the present invention are linear, i.e. unbranched alkanes, whether they be of chain length C8 to C16, C10 to C16 or C12 to C16.

Preferred alkanes are liquids under ambient conditions.

The purpose of the ethanol is principally to solubilise the lipid mixture and the linear fatty alcohol.

The content of ethanol in the total composition, ignoring the propellant therein, is preferably at least 10%, more preferably at least 15% and most preferably at least 30%. The content of ethanol, again ignoring and propellant in the composition, is preferably 90% or and more preferably 85% or less, these upper amounts being combinable with each of the lower ranges indicated in this paragraph to give preferred ranges of incorporation.

Preferred composition according to the invention are aerosol compositions. Such compositions typically employ a volatile propellant. The propellant is commonly either a compressed gas or a material that boils at below ambient temperature, preferably at below 0° C., and especially at below −10° C. Examples of compressed gasses include compressed air, nitrogen and carbon dioxide. Examples of suitable propellants include volatile hydrocarbons, dimethyl ether and hydrofluorocarbons containing from 2 to 4 carbons, at least one hydrogen and 3 to 7 fluorine atoms. In particularly preferred embodiments, the propellant used comprises or is solely a hydrocarbon propellant.

Preferred hydrocarbons for use as propellant include propane, butane, isobutane and mixtures thereof.

When compositions according to the invention are aerosol compositions, they can be made in a conventional manner by first preparing a base composition, charging the base composition into the aerosol can, fitting a valve assembly into the mouth of the can, thereby sealing the can, and thereafter charging propellant into the can to a desired pressure, and finally fitting an actuator on or over the valve assembly.

When employed, propellant is included in the total composition at a level of from 20 to 95% and preferably at a level of from 50 to 90%.

Cosmetic compositions according to the present invention are preferably free from aluminium or zirconium salts.

A preferred additional component for use in compositions of the present invention is a fragrance or fragrance oil, sometimes alternatively called a perfume (oil). The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the composition is commonly up to 3% advantageously is at least 0.5% and particularly from 0.8% to 2%.

A preferred additional component of compositions of the invention is a deodorant active. These are typically antimicrobial agents active against bacterial on the skin of the human body. These serve to reduce malodour and especially useful in compositions in which the lipid mixture is not itself an antimicrobial agent. When employed, the level of incorporation is preferably 0.01%-5%, more preferably from 0.01-2% and most preferably from 0.03%-0.5% by weight of the total composition.

Preferred anti-microbial deodorant agents are those that are more efficacious than simple alcohols such as ethanol. Particularly preferred anti-microbial deodorant agents are soluble in ethanol, meaning that they a solubility in ethanol of at least 10 g/L at 20° C.

Examples of suitable anti-microbial deodorant agents include niacinamide; quaternary ammonium compounds, like cetyltrimethylammonium salts; chlorhexidine and salts thereof; and diglycerol monocaprate, diglycerol monolaurate, and similar materials, as described in “Deodorant Ingredients”, S. A. Makin and M. R. Lowry, in “Antiperspirants and Deodorants”, Ed. K. Laden (1999, Marcel Dekker, New York). More preferred are polyhexamethylene biguanide salts (also known as polyaminopropyl biguanide salts), an example being Cosmocil CQ available from Arch Chemicals, 2′,4,4′-trichloro,2-hydroxy-diphenyl ether (triclosan), 3,7,11-trimethyldodeca-2,6,10-trienol (farnesol), essential oils such as Tea Tree Oil and Thyme Oil, climbazole, octapyrox, ketoconazole, zinc pyrithione and mixtures thereof.

A preferred optional component is a preservative, such as ethyl or methyl parabens or BHT (butyl hydroxy toluene), typically in an amount of from 0.01 to 0.1% by weight of the total composition.

The invention will now be described by way of some examples, which do not limit the extent of the invention.

### Examples

The following raw materials were used in the preparation of the Examples:
Glycerol monolaurate; Monomuls 90-L12, ex BASF.
Isostearyl alcohol; Prisorine 3515, ex Croda.
Denatured alcohol, ex VWR.
Lanette 18 DEO, stearyl alcohol, ex BASF
Lanette O, a blend of cetyl alcohol and stearyl alcohol, ex BASF. (45-55% C16, 45-55% C18, <3% C14, <3% C20).
Lanette 16, cetyl alcohol, ex BASF. (>95% C16, <3% C14, <5% C18).
Tetradecanol, 1-tetradecanol, ex Aldrich.
Dodecanol, 1-dodecanol, ex Aldrich.
Decanol, 1-decanol, ex Aldrich.

Several mixtures of lipid and linear fatty alcohol (or alternatives thereto) were prepared by heating the mixtures to 60-80° C. to ensure the solids were molten and allow good mixing.

The lipid used was blend of GML and ISA at a weight ratio of 3:1). The mixtures so prepared were applied to microscope slides and, following cooling to 37° C., were examined for the presence of inverse hexagonal phase using polarised light microscopy. The mixtures tested and results obtained are illustrated in the following Tables.

The amounts of components indicated were those used to two decimal places and the ratios indicated are to the nearest whole number, rounding up in favour of the major component when relevant.

The samples involved in this test mimic the balance of linear alcohol, lipid and water that would exist on the surface of the human body following secretion of sweat (which has water as major component) and the application of a composition according to the present invention. For compositions according to the invention comprising ethanol, it may be assumed that the ethanol would evaporate, leaving the linear alcohol, lipid and water from the sweat as the components present on the surface of the human body.

TABLE 1

| (b) Linear fatty alcohol | | (a) Lipid | Water | (a):(b) | Inverse |
|---|---|---|---|---|---|
| Identity | wt. % | wt. % | wt. % | wt. ratio | hexagonal? |
| Stearyl | 10 | 50 | 40 | 83:17 | No |
| alcohol | 10 | 60 | 30 | 86:14 | Yes |
| | 10 | 65 | 25 | 87:13 | Yes |
| | 10 | 70 | 20 | 88:12 | Yes |
| | 10 | 75 | 15 | 88:12 | Yes |
| | 10 | 80 | 10 | 89:11 | No |
| | 15 | 60 | 25 | 80:20 | Yes |
| | 15 | 65 | 20 | 81:19 | Yes |
| | 15 | 70 | 15 | 82:18 | Yes |
| | 20 | 65 | 15 | 77:23 | No |
| | 25 | 60 | 15 | 71:29 | No |
| Lanette O | 10 | 50 | 40 | 83:17 | No |
| | 10 | 60 | 30 | 86:14 | Yes |
| | 10 | 65 | 25 | 87:13 | Yes |
| | 10 | 70 | 20 | 88:12 | Yes |
| | 10 | 75 | 15 | 88:12 | Yes |
| | 10 | 80 | 10 | 89:11 | No |
| | 15 | 60 | 25 | 80:20 | Yes |
| | 15 | 65 | 20 | 81:19 | Yes |
| | 15 | 70 | 15 | 82:18 | Yes |
| | 20 | 65 | 15 | 77:23 | Yes |
| | 25 | 60 | 15 | 71:29 | No |

Whilst the “10, 50, 40” samples in the top lines for the stearyl alcohol and Lanette 0 entries did not form inverse hexagonal phase, it will be noted that samples having lower water content, but comparable (a):(b) ratios (above and below), did do so. Hence, it is clear that these samples, if concentrated somewhat, would have been capable of forming inverse hexagonal phase.

TABLE 2

| (b) Linear fatty alcohol | | (a) Lipid | Water | (a):(b) | Inverse |
|---|---|---|---|---|---|
| Identity | wt. % | wt. % | wt. % | wt. ratio | hexagonal? |
| Lanette 16 | 10 | 50 | 40 | 83:17 | Yes |
| | 10 | 60 | 30 | 86:14 | Yes |
| | 10 | 65 | 25 | 87:13 | Yes |
| | 10 | 70 | 20 | 88:12 | Yes |
| | 10 | 75 | 15 | 88:12 | Yes |
| | 10 | 80 | 10 | 89:11 | No |
| | 15 | 60 | 25 | 80:20 | No |
| | 15 | 65 | 20 | 81:19 | Yes |
| | 15 | 70 | 15 | 82:18 | Yes |
| | 20 | 65 | 15 | 77:23 | Yes |
| | 25 | 60 | 15 | 71:29 | No |
| Tetradecanol | 10 | 50 | 40 | 83:17 | Yes |
| | 10 | 60 | 30 | 86:14 | Yes |
| | 10 | 65 | 25 | 87:13 | Yes |
| | 10 | 70 | 20 | 88:12 | Yes |
| | 10 | 75 | 15 | 88:12 | Yes |
| | 10 | 80 | 10 | 89:11 | No |
| | 15 | 60 | 25 | 80:20 | Yes |
| | 15 | 65 | 20 | 81:19 | Yes |
| | 15 | 70 | 15 | 82:18 | Yes |
| | 20 | 65 | 15 | 77:23 | Yes |
| | 25 | 60 | 15 | 71:29 | No |

Whilst the “15, 60, 25” sample in the Lanette 16 entry did not form inverse hexagonal phase, it will be noted that samples having lower water content, but comparable (a):(b) ratios (above and below), did do so. Hence, it is clear that this sample, if concentrated somewhat, would have been capable of forming inverse hexagonal phase.

TABLE 3

| (b) Linear fatty alcohol | | (a) Lipid | Water | (a):(b) | Inverse |
|---|---|---|---|---|---|
| Identity | wt. % | wt. % | wt. % | wt. ratio | hexagonal? |
| Dodecanol | 10 | 50 | 40 | 83:17 | No |
| | 10 | 60 | 30 | 86:14 | Yes |
| | 10 | 65 | 25 | 87:13 | Yes |
| | 10 | 70 | 20 | 88:12 | Yes |
| | 10 | 75 | 15 | 88:12 | Yes |
| | 10 | 80 | 10 | 89:11 | No |
| | 15 | 60 | 25 | 80:20 | No |
| | 15 | 65 | 20 | 81:19 | Yes |
| | 15 | 70 | 15 | 82:18 | Yes |
| | 20 | 65 | 15 | 77:23 | No |
| | 25 | 60 | 15 | 71:29 | No |
| Decanol | 10 | 50 | 40 | 83:17 | No |
| | 10 | 60 | 30 | 86:14 | Yes |
| | 10 | 65 | 25 | 87:13 | Yes |
| | 10 | 70 | 20 | 88:12 | Yes |
| | 10 | 75 | 15 | 88:12 | Yes |
| | 10 | 80 | 10 | 89:11 | No |
| | 15 | 60 | 25 | 80:20 | No |
| | 15 | 65 | 20 | 81:19 | Yes |
| | 15 | 70 | 15 | 82:18 | Yes |
| | 20 | 65 | 15 | 77:23 | No |
| | 25 | 60 | 15 | 71:29 | No |

Whilst the “10, 50, 40” sample in the top line for decanol entry did not form inverse hexagonal phase, it will be noted that samples having lower water content, but comparable (a):(b) ratios (above and below) did do so. Hence, it is clear that this sample, if concentrated somewhat, would have been capable of forming inverse hexagonal phase.

Some further tests were performed using linear alkanes instead of linear fatty alcohols, the results from which are shown in Tables 4 and 5. Linear alkanes were found to be somewhat less successful in terms of enabling the lipid to still form inverse hexagonal phase, although there were some successes. Surprisingly, the scope of success was similar to that found for stearyl alcohol.

TABLE 4

| (b) Linear alkane | | (a) Lipid | Water | (a):(b) | Inverse |
|---|---|---|---|---|---|
| Identity | wt. % | wt. % | wt. % | wt. ratio | hexagonal? |
| Octadecane | 10 | 50 | 40 | 83:17 | No |
| | 10 | 65 | 25 | 87:13 | No |
| | 10 | 80 | 10 | 89:11 | No |
| | 15 | 70 | 15 | 82:18 | No |
| | 20 | 55 | 25 | 73:27 | No |
| | 30 | 60 | 10 | 67:33 | No |
| Hexadecane | 10 | 50 | 40 | 83:17 | No |
| | 10 | 55 | 35 | 85:15 | No |
| | 10 | 65 | 25 | 87:13 | Yes |
| | 10 | 75 | 15 | 88:12 | No |
| | 10 | 80 | 10 | 89:11 | No |
| | 20 | 65 | 15 | 77:23 | No |
| | 30 | 60 | 10 | 67:33 | No |

TABLE 5

| (b) Linear alkane | | (a) Lipid | Water | (a):(b) | Inverse |
|---|---|---|---|---|---|
| Identity | wt. % | wt. % | wt. % | wt. ratio | hexagonal? |
| Tetradecane | 10 | 50 | 40 | 83:17 | No |
| | 10 | 55 | 35 | 85:15 | Yes |
| | 10 | 65 | 25 | 87:13 | Yes |
| | 10 | 75 | 15 | 88:12 | No |
| | 10 | 80 | 10 | 89:11 | No |
| | 20 | 65 | 15 | 77:23 | No |
| | 30 | 60 | 15 | 67:33 | No |
| Dodecane | 10 | 50 | 40 | 83:17 | Yes |
| | 10 | 65 | 25 | 87:13 | Yes |
| | 10 | 75 | 15 | 88:12 | No |
| | 10 | 80 | 10 | 89:11 | No |
| | 20 | 50 | 30 | 71:29 | No |
| | 20 | 65 | 15 | 77:23 | No |
| | 25 | 60 | 15 | 71:29 | No |

Further comparative tests were performed using other alternative fatty materials, instead of linear fatty alcohols. The other alternative was mixed with the lipid blend in the same way as described in the studies above. The following alternative materials failed to give inverse hexagonal phase with the lipid blend and water, across multiple ratios:
Eutanol G, 2-octyldodecanol, ex BASF.
Lexfeel 7, neopentyl glycol diheptanoate, Inolex.
Bees wax.
Laurel wax.
Capric/caprylic triglycerides.

The Examples according to the invention shown in Table 6 were prepared using methods known in the art. The stability of these aerosol compositions and their corresponding base compositions ( . . . B) were assessed by storage at 0° C. and 20° C. over a period of 12 wks. Instability was manifested by crystallisation of materials out of the indicated mixture. The Examples and results are shown in the Table.

It will be noted that each of total [i.e. aerosol compositions were stable at both 0° C. and 20° C. for the period of the test. The base compositions were also stable at 20° C., but not at 0° C.

TABLE 6

| | Example: | | | | | | | |
|---|---|---|---|---|---|---|---|---|
| | 1B | 1 | 2B | 2 | 3B | 3 | 4B | 4 |
| | Wt. % | Wt. % | Wt. % | Wt. % | Wt. % | Wt. % | Wt. % | Wt. % |
| Component | base | total | base | total | base | total | base | total |
| Glyceryl monolaurate | 12.10 | 6.05 | 12.10 | 6.05 | 12.10 | 6.05 | 12.10 | 6.05 |
| Isopropyl alcohol | 4.03 | 2.02 | 4.03 | 2.02 | 4.03 | 2.02 | 4.03 | 2.02 |
| Stearyl alcohol (C18) | 2.31 | 1.16 | — | — | — | — | — | — |
| Cetyl alcohol (C16) | — | — | 2.31 | 1.16 | — | — | — | — |
| Myristyl alcohol (C14) | — | — | — | — | 2.31 | 1.16 | — | — |
| Lauryl alcohol (C12) | — | — | — | — | — | — | 2.31 | 1.16 |
| Ethanol | 81.56 | 40.78 | 81.56 | 40.78 | 81.56 | 40.78 | 81.56 | 40.78 |
| Propellant (AP40) | — | 50.00 | — | 50.00 | — | 50.00 | — | 50.00 |
| Stable at 0° C.? | No | Yes | No | Yes | No | Yes | No | Yes |
| Stable at 20° C.? | Yes | Yes | Yes | Yes | Yes | Yes | Yes | Yes |

1. A cosmetic aerosol composition comprising:
(a) a lipid mixture consisting of isostearyl alcohol and glycerol monolaurate at a weight ratio of from 25:75 to 40:60 at a level of from 2.5% to 25% by weight; and
(b) a fatty material selected from C10-C18 linear;
wherein the weight ratio of component (a) to total components (b) is from 73:27 to 88:12 by weight; with the additional requirements that:
(j) when 1-decanol and/or 1-dodecanol is the major component (b) present, the ratio of component (a) to component (b) is from 81:19 to 88:12;
(ii) when stearyl alcohol is the major component (b) present, the ratio of component (a) to component (b) is from 78:22 to 88:12;
wherein silicone oil is absent from the composition and wherein the amounts indicated exclude any volatile propellant present in the composition.

2. A composition according to claim 1, comprising ethanol at a level of from 10 to 90% by weight of the composition, excluding any volatile propellant therein.

3. A composition according to claim 2, comprising ethanol at a level of from 30 to 85% by weight of the composition, excluding any volatile propellant therein.

4. A composition according to claim 1, comprising volatile propellant at a level of from 20 to 95% by weight of the total composition.

5. A composition according to claim 4, comprising volatile propellant at a level of from 50 to 90%.

6. A composition according to claim 1 in the form of a sprayable solution packaged in a spray dispenser.

7. A composition according to claim 1, wherein the content of the lipid mixture in the total composition, excluding any volatile propellant therein, is at least 10% by weight.

8. A composition according to claim 7, wherein the content of the lipid mixture in the total composition, ignoring the propellant therein, is at least 15% by weight.

9. (canceled)

10. A composition according to claim 1, which is free from aluminium or zirconium salts.

11. A composition according to claim 1, comprising an antimicrobial agent.

12. (canceled)

13. (canceled)

14. A cosmetic method of reducing wetness perception comprising the topical application of a composition according to claim 1.
